(19) United States
(12) Patent Application Publication
(10) Pub. No.: US 2011/0319845 A1
(43) Pub. Date: Dec. 29, 2011

(54) Diaper structure with urine sensor

(75) Inventors: You-Ti Kuo, Penfield, NY (US); Sharon Lin Charna, Irvine, CA (US); Matthew Chang, Irvine, CA (US); Lee-Tsan Lu, Taipei City (TW)

(73) Assignee: At Box Technology Inc., Irvine, CA (US)

(21) Appl. No.: 13/064,832

(22) Filed: Apr. 20, 2011

Related U.S. Application Data
(60) Provisional application No. 61/398,563, filed on Jun. 28, 2010.

(30) Foreign Application Priority Data
Nov. 15, 2010 (TW) 099222074

(51) Int. Cl. A61F 13/42

(52) U.S. Cl. 604/361

(57) ABSTRACT

The present invention is to provide a diaper structure including two electrode strips, wherein the two electrode strips are disposed inside the diaper at a bottom position thereof and electrically connected to each other when both in contact with urine, and two metal button seats are disposed inside the diaper and are electrically connected to the two electrode strips respectively. Each of the two metal button seats has a first fastening portion disposed on the outside surface of the diaper and fastened with an electrical detection device, so that the two metal button seats are electrically connected to the electrical detection device via the two first fastening portions respectively for enabling the electrical detection device to send out a wetness signal when the two electrode strips are electrically connected to each other due to in contact with urine, thereby urging the caregiver to change the diaper structure immediately.

### FIELD OF THE INVENTION

The present invention relates to a diaper structure with a urine sensor, which includes two electrode strips. The two electrode strips are attached to a bottom side of a first fabric layer of the diaper and electrically connected to each other when in contact with urine. Two metal button seats are provided within the diaper, and correspond in position to and are electrically connected to the two electrode strips respectively. Moreover, each of the two metal button seats has a first fastening portion disposed on the outside surface of the diaper and fastened with an electrical detection device, so that the two metal button seats are electrically connected to the electrical detection device for enabling the electrical detection device to detect whether the two electrode strips are electrically connected to each other and send out a wetness signal when the two electrode strips are electrically connected to each other due to in contact with urine, thereby urging the caregiver to change the diaper structure immediately.

### BACKGROUND OF THE INVENTION

Referring to FIG. 1 and FIG. 2, a product 10 (e.g., diaper) capable of urine detection mainly includes a first nonwoven fabric layer 11, an absorbent layer 12, a second nonwoven fabric layer 13, two sensing electrodes 14, and a plastic layer 15. The first nonwoven fabric layer 11 is configured to allow urine discharged from the human body to quickly flow therethrough from a first side to a second side of the first nonwoven fabric layer 11. The absorbent layer 12 is composed of an absorbent material and has a first side attached to the second side of the first nonwoven fabric layer 11 and is configured to absorb the urine passing through the first nonwoven fabric layer 11 so that the urine is in turn absorbed and kept in the absorbent layer 12. The second nonwoven fabric layer 13 has a first side attached to both the second side of the first nonwoven fabric layer 11 and a second side of the absorbent layer 12. Thus, the absorbent layer 12 is sandwiched and secured between the first nonwoven fabric layer 11 and the second nonwoven fabric layer 13. The second nonwoven fabric layer 13 is configured to allow the urine leaking out of the absorbent layer 12 to flow therethrough from the first side to a second side of the second nonwoven fabric layer 13. The two sensing electrodes 14 are electrode strips, each having a first side attached to the second side of the second nonwoven fabric layer 13. The two sensing electrodes 14 are electrically connected to each other upon contact with urine. The plastic layer 15 has a first side attached to both the second side of the second nonwoven fabric layer 13 and a second side of each sensing electrode 14 such that the two sensing electrodes 14 are sandwiched and secured between the second nonwoven fabric layer 13 and the plastic layer 15. The plastic layer 15 is configured to hold the urine leaking out of the second nonwoven fabric layer 13 and prevent such urine from leaking out of the product 10. Once the first nonwoven fabric layer 11, the absorbent layer 12, the second nonwoven fabric layer 13, the sensing electrodes 14, and the plastic layer 15 are sequentially sealed together to form a single piece, the two sensing electrodes 14 are positioned in the product 10 and yet each sensing electrode 14 has one end exposed outside the product 10.

A caregiver of a disabled senior or of a baby (the latter two generally referred to herein as “the user”) can wrap the product 10 around the user's pelvic region and, as shown in FIG. 2, fasten the two alligator clips 17 of the clip connector 18 of an electrical detection device 16 to the exposed ends of the two sensing electrodes 14 respectively. Thus, when the user urinates, the urine passes through the first nonwoven fabric layer 11 to the absorbent layer 12 and is stored therein. Once the amount of urine stored in the absorbent layer 12 exceeds the absorbing capacity of the absorbent layer 12, the urine will leak through the second side of the absorbent layer 12 and be absorbed by the second nonwoven fabric layer 13. As soon as the amount of urine absorbed by the second nonwoven fabric layer 13 reaches a certain level, the two sensing electrodes 14 will come into contact with the urine and hence be electrically connected to each other, allowing the electrical detection device 16 to detect the electrically connected state of the two sensing electrodes 14 via the two alligator clips 17 of the clip connector 18 and give out a warning sound accordingly. The warning sound alerts the caregiver to the fact that the urine in the product 10 has reached a predetermined amount and that it is time to clean the user and change the product 10 for a new one.

However, the product 10 capable of urine detection as described above has its drawbacks in use. First of all, as the sensing electrodes 14 in the product 10 are enclosed between the second nonwoven fabric layer 13 and the plastic layer 15 and are located near the bottom of the product 10, electrical connection between the two sensing electrodes 14 due to physical contact with the urine in the second nonwoven fabric layer 13 will not take place—and the electrical detection device 16 will not give off the warning sound—until both the first nonwoven fabric layer 11 and the absorbent layer 12 are saturated with urine and the second nonwoven fabric layer 13 has absorbed a certain amount of urine that leaks from the absorbent layer 12. Thus, the sensitivity of urine detection by the product 10 is compromised. In other words, only when a large amount of urine is accumulated in the product 10 will the electrical detection device 16 be triggered to generate a sound alert. Under such circumstances, the user's skin in the pelvic region will be in contact with the urine-saturated first nonwoven fabric layer 11 for a prolonged period of time and hence vulnerable to skin irritation or even inflammation. As a result, not only is the user made uncomfortable, but also the caregiver will find more difficulty in subsequent caretaking of the user.

Secondly, the product 10 capable of urine detection is configured in such a way that the two sensing electrodes 14 are exposed at one end thereof so that the two alligator clips 17 of the clip connector 18 of the electrical detection device 16 can be fastened to the exposed ends of the sensing electrodes 14 respectively. However, as the clip connector 18 and the alligator clips 17 have rather complicated structures and large volumes, the user, when lying in bed wearing the product 10, is very likely to have his or her body pressed against the clip connector 18 and the alligator clips 17, thus causing an uncomfortable sensation. In addition, the bulky clip connector 18 and alligator clips 17 are clipped only on the exposed ends of the sensing electrodes 14 and are therefore subject to displacement when accidentally pushed or pulled by the user. Should the alligator clips 17 be moved out of position, the electrical detection device 16 will be unable to detect urine accurately, and skin irritation or inflammation may result from long-term contact with urine as previously mentioned.

Therefore, the issue to be addressed by the present invention is to solve the problems of the foregoing product capable of urine detection. These problems include: skin irritation caused by extended contact with urine as a result of an insensitive urine detection design that places the sensing electrodes at the bottom of the product and therefore tends to bring about excessive accumulation of urine; discomfort arising from the user's body pressing against the bulky alligator clips that are clipped on the sensing electrodes in order for the product to detect urine and; frequent displacement of the alligator clips due to unintentional push or pull by the user. The present invention aims to design a diaper structure with a urine sensor, wherein urine, once discharged, can be immediately sensed by electrode strips provided below a first fabric layer that serves as the top layer of the diaper structure, wherein an electrical detection device can detect urine rapidly by means of a button-type connection between button-shaped elements of the electrical detection device and metal button seats provided at the bottom of the diaper structure, and wherein the electrical detection device, upon detecting urine, gives off a wetness signal to urge the caregiver to change the diaper structure for a new one.

### BRIEF SUMMARY OF THE INVENTION

In view of and in order to effectively solve the problems of the aforementioned conventional product capable of urine detection—namely a low sensitivity in urine detection that often leads to an excessive accumulation of urine, and the use of alligator clips, which can be easily pulled out of place or cause discomfort when pressed against by the user's body—the inventor of the present invention made great efforts in research and experiment and finally succeeded in developing a diaper structure with a urine sensor as disclosed herein.

It is an object of the present invention to provide a diaper structure with a urine sensor, wherein the diaper structure is configured to be wrapped around the pelvic region of a human body and prevent urine or feces from leaking. The diaper structure includes a first fabric layer, two electrode strips, a second fabric layer, an absorbent material layer, an anti-leak layer, and two metal button seats. The first fabric layer is made of a nonwoven fabric and is configured to allow urine discharged from the human body to quickly flow therethrough from a first side to a second side of the first fabric layer. The two electrode strips, each having a first side attached to the second side of the first fabric layer, are electrically connected to each other when in contact with urine. The second fabric layer is made of a nonwoven fabric and has a first side attached to both the second side of the first fabric layer and a second side of each of the two electrode strips such that the two electrode strips are sandwiched and secured between the first fabric layer and the second fabric layer. The second fabric layer is configured to allow the urine passing through the first fabric layer to flow therethrough from the first side to a second side of the second fabric layer. The absorbent material layer is made of an absorbent material (e.g., pulp, cotton fibers, and sodium polyacrylate) and has a first side attached to the second side of the second fabric layer and is configured to absorb the urine passing through the second fabric layer so that the urine is in turn absorbed and kept in the absorbent material layer. The anti-leak layer is made of a waterproof plastic and has a first side attached to a second side of the absorbent material layer. The first side of the anti-leak layer is configured to hold the urine leaking out of the absorbent material layer and prevent the urine from leaking out of the diaper structure. The anti-leak layer, the second fabric layer, and the first fabric layer are sequentially sealed together at their peripheries to form a single piece, thereby securing the electrode strips and the absorbent material layer at corresponding positions. The two metal button seats are secured on a second side of the anti-leak layer and each correspond in position to one end of one of the two electrode strips. In addition, the two metal button seats pierce through the anti-leak layer so as to be electrically connected to the ends of the two electrode strips respectively. Moreover, each of the two metal button seats has a first fastening portion. The two first fastening portions can be fastened respectively with the second fastening portions of two button-shaped elements of an electrical detection device so that the two metal button seats are electrically connected to the electrical detection device via the corresponding button-shaped elements.

It is another object of the present invention to provide the foregoing diaper structure, wherein the diaper structure, when wrapped around the pelvic region of a disabled user (e.g., an elderly or an infant), allows the urine discharged from the user to pass rapidly through the first fabric layer and the second fabric layer to the absorbent material layer and be kept therein. While the urine passes from the first fabric layer to the second fabric layer, the two electrode strips contact with the urine and are thus electrically connected to each other. In consequence, by way of the fastened together and electrically connected button-shaped elements and metal button seats, the electrical detection device detects the electrically connected state of the two electrode strips. Then, the electrical detection device sends out a wetness signal in a wired or wireless manner to alert the caregiver to the fact that the user has urinated, thereby urging the caregiver to change the diaper structure immediately. Hence, the user is protected from skin irritation in the pelvic area that is attributable to long-term contact with urine as in the case of the prior art, where the sensitivity of urine detection is lowered by placing the sensing electrodes on the bottom layer of the product such that an excessive accumulation of urine tends to take place. Furthermore, as the disclosed diaper structure completely envelopes the electrode strips, and electrical connection between the electrode strips and the electrical detection device is established by the relatively small metal button seats and button-shaped elements fastened together, the bulky alligator clips which are required in the prior art for urine detection by the electrical detection device and are clipped on the exposed ends of the sensing electrodes can be dispensed with. Therefore, while lying in bed, the user is prevented from the discomfort which may otherwise result from the user's body pressing against the clip connector and the alligator clips. Moreover, since the risks of accidentally pushing or pulling and hence displacing the bulky clip connector and alligator clips are eliminated, the accuracy of urine detection by the electrical detection device is ensured.

### BRIEF DESCRIPTION OF THE SEVERAL VIEWS OF THE DRAWINGS

The structure as well as a preferred mode of use, further objects, and advantages of the present invention will be best understood by referring to the following detailed description of some illustrative embodiments in conjunction with the accompanying drawings, in which:

FIG. 1 is an exploded perspective view of a product capable of urine detection;

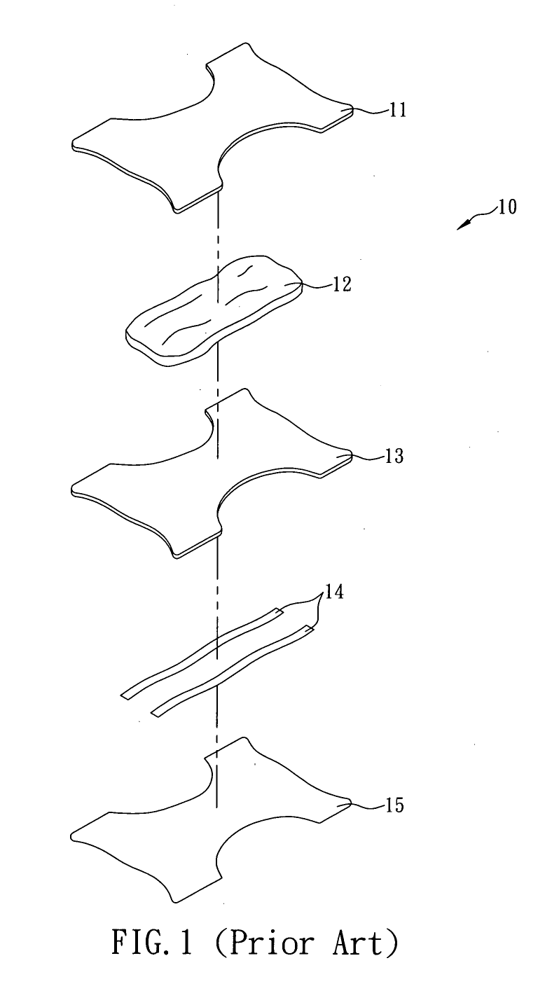

FIG. 2 is another perspective view of the product depicted in FIG. 1, showing in addition an electrical detection device to be connected to the product;

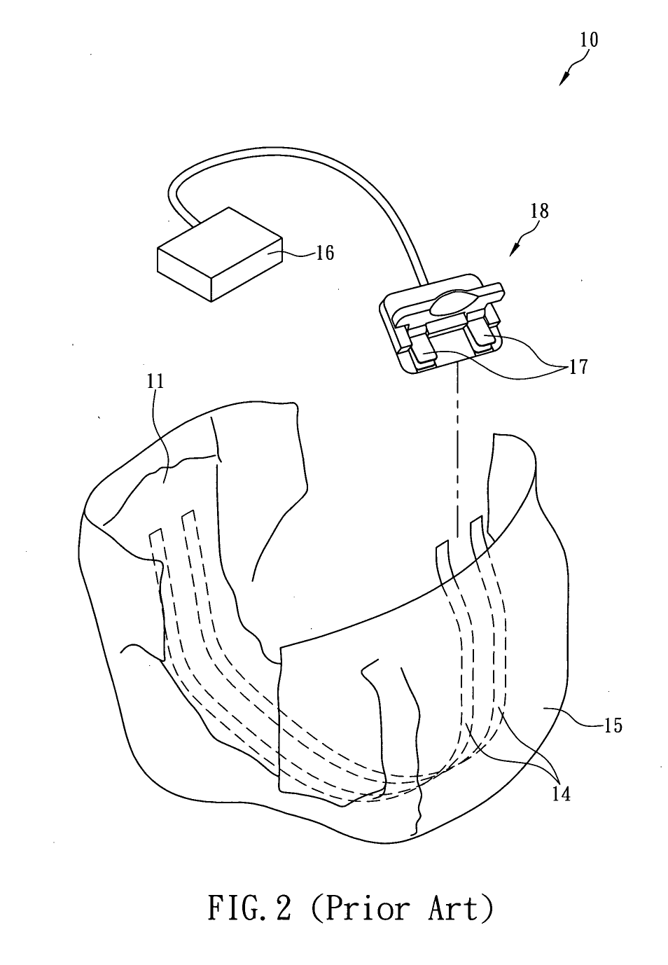

FIG. 3 is an exploded perspective view of a first preferred embodiment of the present invention;

FIG. 4 is another perspective view of the first preferred embodiment of the present invention, showing in addition an electrical detection device to be connected thereto; and

FIG. 5 is an exploded perspective view of a second preferred embodiment of the present invention.

### DETAILED DESCRIPTION OF THE INVENTION

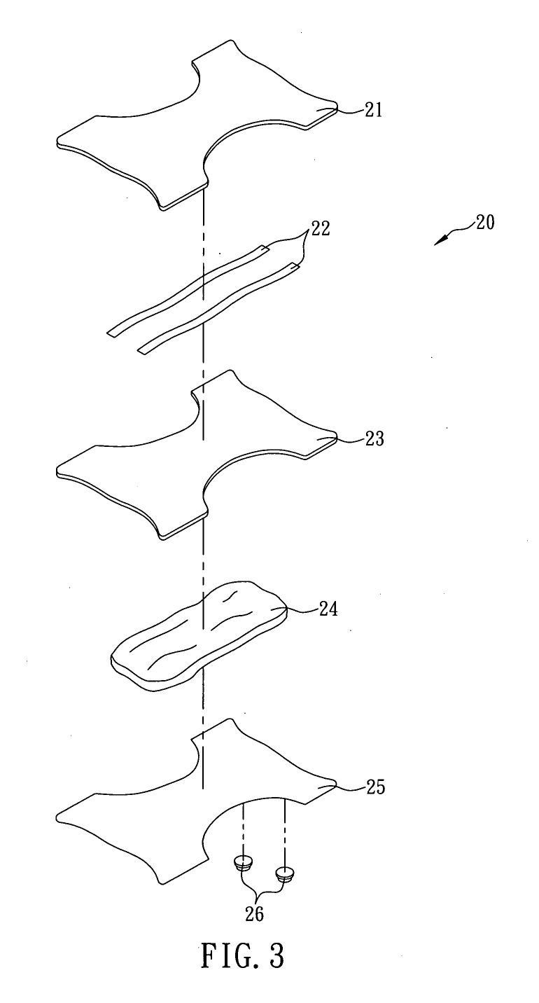

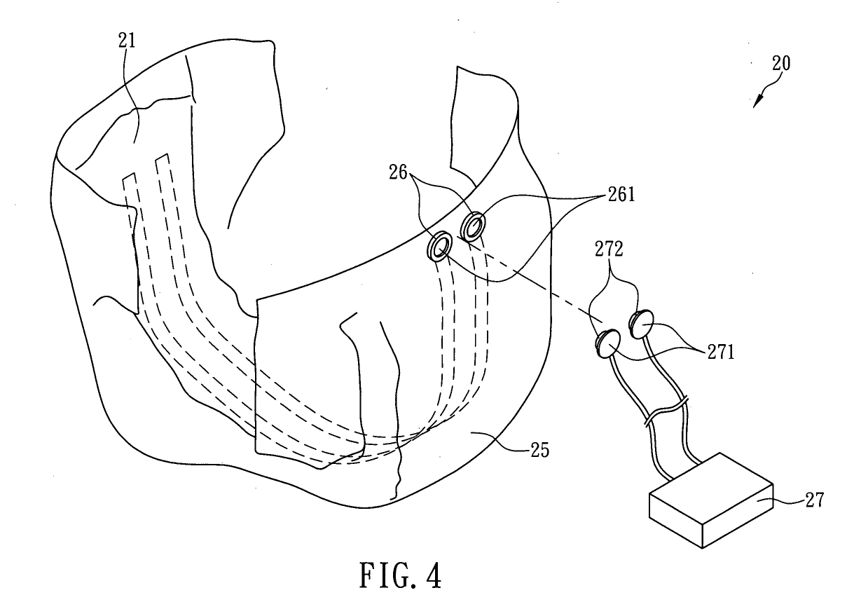

The present invention provides a diaper structure with a urine sensor. Referring to FIGS. 3 and 4, a diaper structure 20 according to a first preferred embodiment of the present invention includes a first fabric layer 21, two electrode strips 22, a second fabric layer 23, an absorbent material layer 24, an anti-leak layer 25, and two metal button seats 26. The first fabric layer 21, made of a nonwoven fabric, is configured to allow urine discharged from the human body to quickly flow therethrough from a first side to a second side of the first fabric layer 21. The two electrode strips 22, each having a first side attached to the second side of the first fabric layer 21, are spaced from each other and, when in contact with urine, become electrically, connected to each other. The second fabric layer 23, also made of a nonwoven fabric, has a first side covering both the second side of the first fabric layer 21 and a second side of each of the two electrode strips 22; consequently, the two electrode strips 22 are sandwiched and secured between the first fabric layer 21 and the second fabric layer 23. The second fabric layer 23 is configured to allow the urine passing through the first fabric layer 21 to flow therethrough from the first side to a second side of the second fabric layer 23.

With reference to FIGS. 3 and 4, the absorbent material layer 24 is formed of a mixture of absorbent materials, such as pulp, cotton fibers, and a superabsorbent polymer (SAP), wherein the superabsorbent polymer includes sodium polyacrylate and is capable of absorbing urine over sixty times its own weight. The absorbent material layer 24 has a first side attached to the second side of the second fabric layer 23 and is configured to absorb the urine passing through the second fabric layer 23 so that the urine is in turn absorbed and kept in the absorbent material layer 24. The anti-leak layer 25 is made of a waterproof plastic and has a first side attached to a second side of the absorbent material layer 24. The first side of the anti-leak layer 25 is configured to hold the urine leaking out of the absorbent material layer 24 and prevent the urine from leaking out of the diaper structure 20. The anti-leak layer 25, the second fabric layer 23, and the first fabric layer 21 are sequentially sealed together along their peripheries to form a single piece and thereby fix the two electrode strips 22 and the absorbent material layer 24 at corresponding positions. The two metal button seats 26 are secured on a second side of the anti-leak layer 25, and each metal button seat 26 corresponds in position to one end of one of the two electrode strips 22. In addition, the two metal button seats 26 pierce through the anti-leak layer 25 so as to form electrical connection with the ends of the two electrode strips 22 respectively. Each of the two metal button seats 26 has a first fastening portion 261. The two first fastening portions 261 can be fastened with the second fastening portions 272 of two button-shaped elements 271 of an electrical detection device 27 respectively so that the two metal button seats 26 are electrically connected to the electrical detection device 27 via the corresponding button-shaped elements 271.

In the present embodiment, each first fastening portion 261 is in the form of a hole, and each second fastening portion 272 is in the form of a flange. The second fastening portions 272 correspond in configuration to and can be fastened with the first fastening portions 261 respectively, thereby connecting the button-shaped elements 271 and the metal button seats 26 physically and electrically. In practice, however, the second fastening portions 272 and the first fastening portions 261 can be holes and flanges respectively. The configurations of the first and second fastening portions 261, 272 of the present invention are not limited to those disclosed herein and may include all those matching configurations that can be fastened with each other.

When a disabled user (e.g., an elderly or an infant) wearing the diaper structure 20 of the present invention urinates, the diaper structure 20 allows the urine to pass rapidly through the first fabric layer 21 and the second fabric layer 23 to the absorbent material layer 24 and be kept therein. While the urine is delivered from the first fabric layer 21 to the second fabric layer 23, the two electrode strips 22 contact with the urine and are thus electrically connected to each other. As a result, the electrical detection device 27 detects the electrically connected state of the two electrode strips 22 via the button-shaped elements 271 and the metal button seats 26 that are fastened together and electrically connected. Then, the electrical detection device 27 sends out a wetness signal in a wired or wireless manner to notify the caregiver that the user has urinated and thereby urge the caregiver to change the diaper structure 20 immediately. As the two electrode strips 22 are not located on the bottom layer of the diaper structure 20 as in the prior art, the sensitivity of urine detection will not be compromised, and urine is prevented from accumulating in the diaper structure 20 to such an excessive extent that the user's skin in the pelvic area will be in contact with the urine for a long time and therefore subject to skin irritation. Moreover, in the present invention, the two electrode strips 22 are completely enclosed in the diaper structure 20, and electrical connection between the two electrode strips 22 and the electrical detection device 27 is realized by the relatively small metal button seats 26 and button-shaped elements 271 that are securely fastened together. This design is significantly different from that of the prior art, where the sensing electrodes are exposed and are connected with relatively large alligator clips so as for the electrical detection device to detect urine. As previously mentioned, the bulky alligator clips cause an uncomfortable sensation to the user when pressed against by the user's body lying in bed and is likely to be moved out of position when accidentally pushed or pulled by the user, thus lowering the accuracy of urine detection.

Furthermore, as the diaper structure 20 is configured to detect urine via the two electrode strips 22 and send out the wetness signal accordingly each time the user urinates, the caregiver can count the number of times the wetness signal is sent by the same diaper structure 20—thus knowing the number of times the user has urinated—and change the diaper structure 20 at an appropriate time. Besides, the electrical conductivity between the two electrode strips 22 depends on the pH value of urine. More specifically, the lower the pH value of the urine is, the higher the sodium ion concentration of the urine will be, and consequently the higher the electrical conductivity between the two electrode strips 22 will be. In the present embodiment, the electrical detection device 27 is configured not only to detect whether the two electrode strips 22 are electrically connected, but also to detect the electrical conductivity between the two electrode strips 22. Based on the electrical conductivity detected, the electrical detection device 27 calculates the pH value of the urine with which the two electrode strips 22 are in contact and thereby allows the caregiver to determine the user's current physical condition (e.g., whether the user suffers from urinary tract inflammation, malnutrition, or skin inflammation).

In the present embodiment, the diaper structure 20 is manufactured in the following manner. To begin with, the first fabric layer 21, the electrode strips 22, the second fabric layer 23, the absorbent material layer 24, and the anti-leak layer 25 are placed on a fully automatic machine. The aforementioned components are sequentially sealed together in a continuous process to form a one-piece semi-finished product. Then, the metal button seats 26 are installed on the semi-finished product to complete the diaper structure 20. In practice, however, it is feasible to seal the two electrode strips 22 in between the first fabric layer 21 and the second fabric layer 23 beforehand such that the first fabric layer 21, the electrode strips 22, and the second fabric layer 23 form a one-piece semi-finished product. After that, the semi-finished product (composed of the first fabric layer 21, the electrode strips 2, and the second fabric layer 23 sealed together), the absorbent material layer 24, and the anti-leak layer 25 are placed on a fully automatic machine and sequentially sealed together in a continuous process. Then, the metal button seats 26 are put in place to conclude the manufacture of the diaper structure 20. The manufacturing process of the diaper structure 20 is not limited to those described above, provided that the diaper structure 20 includes the first fabric layer 21, the two electrode strips 22, the second fabric layer 23, the absorbent material layer 24, the anti-leak layer 25, and the two metal button seats 26.

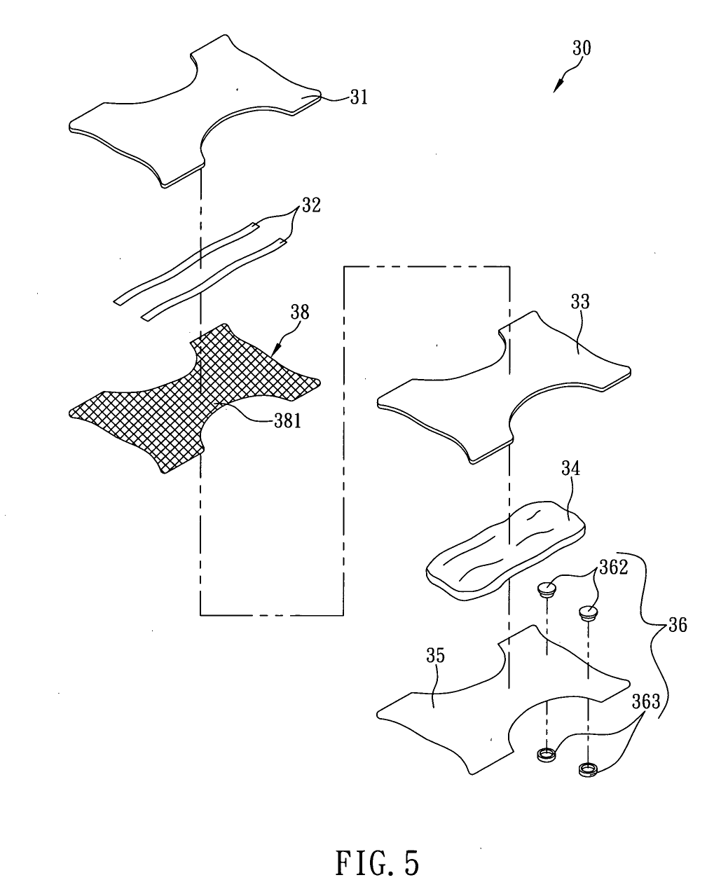

Please refer to FIG. 5 for a second preferred embodiment of the present invention. As shown in the drawing, the diaper structure 30 further includes a plastic mesh layer 38. The plastic mesh layer 38 is made of a waterproof plastic, such as polycarbonate (PC) or acrylonitrile butadiene styrene (ABS), and has a first side penetrated by a plurality of openings 381. The plastic mesh layer 38 is interposed between the two electrode strips 32 and the second fabric layer 33 so that urine leaking through the second side of the first fabric layer 31 can rapidly permeate the second fabric layer 33 through the openings 381. The second fabric layer 33 is made of a nonwoven fabric and has its first side covering the second side of the first fabric layer 31, the second sides of the two electrode strips 32, and a second side of the plastic mesh layer 38; consequently, the two electrode strips 32 are sandwiched and secured between the first fabric layer 31 and the second fabric layer 33. The second fabric layer 33 is configured to allow the urine passing through the first fabric layer 31 to flow therethrough from the first side to the second side of the second fabric layer 33. The absorbent material layer 34 has its first side attached to the second side of the second fabric layer 33 so as to absorb the urine passing through the second fabric layer 33, thus allowing the urine to be absorbed and kept in the absorbent material layer 34. The anti-leak layer 35 is made of a waterproof plastic and has its first side attached to the second side of the absorbent material layer 34 so as to hold the urine leaking out of the absorbent material layer 34 and prevent the urine from leaking out of the diaper structure 30.

In the second preferred embodiment, each of the two metal button seats 36 consists of a base 362 and a ring 363. The bases 362 are secured on the first side of the anti-leak layer 35, and each base 362 corresponds in position to one end of one of the two electrode strips 32. In addition, each base 362 has a flat top surface and is electrically connected to the end of the corresponding electrode strip 32. Each base 362 further has a bottom surface piercing through the anti-leak layer 35 and fastened with the corresponding ring 363. Thus, by fastening the bases 362 with the rings 363 respectively, the metal button seats 36 are formed on the second side of the anti-leak layer 35 and correspond in position to the ends of the two electrode strips 32 respectively. The flat top surfaces of the bases 362 cause no discomfort to the user wearing the diaper structure 30.

While the invention has been described by means of specific embodiments, numerous modifications and variations could be made thereto by those skilled in the art without departing from the scope of the invention set forth in the claims.

What is claimed is:

1. A diaper structure with a urine sensor, comprising:
a first fabric layer made of a nonwoven fabric configured to allow urine to pass through from a first side to a second side of the first fabric layer;
two electrode strips, each the electrode strip having a first side attached to the second side of the first fabric layer, wherein the two electrode strips are spaced from each other and, upon contact with urine, are electrically connected to each other;
a second fabric layer made of a nonwoven fabric and having a first side covering the second side of the first fabric layer and a second side of each the electrode strip such that the two electrode strips are sandwiched and secured between the first fabric layer and the second fabric layer, the second fabric layer being configured to allow urine to pass through from the first side to a second side of the second fabric layer;
an absorbent material layer made of an absorbent material and having a first side attached to the second side of the second fabric layer, the absorbent material layer being configured to absorb urine passing through the second fabric layer so that the urine is absorbed and kept in the absorbent material layer;
an anti-leak layer made of a waterproof plastic and having a first side attached to a second side of the absorbent material layer, the first side of the anti-leak layer being configured to hold urine leaking out of the absorbent material layer and prevent the urine from leaking out of the diaper structure; and
two metal button seats secured on a second side of the anti-leak layer, each the metal button seat corresponding in position to an end of the electrode strip and piercing through the anti-leak layer so as to be electrically connected to the end of the electrode strip, wherein the two metal button seats can be fastened with and be electrically connected to two button-shaped elements of an electrical detection device respectively.

2. The diaper structure of claim 1, wherein each the metal button seat has a first fastening portion, and each the button-shaped element has a second fastening portion, the first fastening portions corresponding in configuration to and being fastenable to the second fastening portions respectively so that the metal button seats are electrically connected to the button-shaped elements respectively.

3. The diaper structure of claim 1, further comprising a plastic mesh layer made of a waterproof plastic, penetrated by a plurality of openings, and interposed between the two electrode strips and the second fabric layer.

4. The diaper structure of claim 2, further comprising a plastic mesh layer made of a waterproof plastic, penetrated by a plurality of openings, and interposed between the two electrode strips and the second fabric layer.

5. The diaper structure of claim 3, wherein each the metal button seat consists of a base and a ring, each the base being secured on the first side of the anti-leak layer, corresponding in position to the end of the electrode strip, having a top surface electrically connected to the end of the electrode strip, and further having a bottom surface piercing through the anti-leak layer and fastened to a corresponding the ring so as to form a the metal button seat on the anti-leak layer.

6. The diaper structure of claim 4, wherein each the metal button seat consists of a base and a ring, each the base being secured on the first side of the anti-leak layer, corresponding in position to the end of the electrode strip, having a top surface electrically connected to the end of the electrode strip, and further having a bottom surface piercing through the anti-leak layer and fastened to a corresponding the ring so as to form a the metal button seat on the anti-leak layer.

7. The diaper structure of claim 5, wherein the anti-leak layer, the second fabric layer, and the first fabric layer are sequentially sealed together along peripheries thereof to form a single piece and thereby secure the two electrode strips and the absorbent material layer at corresponding positions.

8. The diaper structure of claim 6, wherein the anti-leak layer, the second fabric layer, and the first fabric layer are sequentially sealed together, along peripheries thereof to form a single piece and thereby secure the two electrode strips and the absorbent material layer at corresponding positions.
